(19) United States
(12) Patent Application Publication
(10) Pub. No.: US 2024/0184012 A1
(43) Pub. Date: Jun. 6, 2024

(54) SMART OCCUPANCY SENSOR

(71) Applicant: Clinton Mazur, Glastonbury, CT (US)

(72) Inventor: Clinton Mazur, Glastonbury, CT (US)

(21) Appl. No.: 18/552,983

(22) PCT Filed: Mar. 31, 2022
(86) PCT No.: PCT/US2022/022704

§ 371 Date: Sep. 28, 2023

Related U.S. Application Data
(60) Provisional application No. 63/168,486, filed on Mar. 31, 2021.

(51) Int. Cl. G01V 8/20; G05B 15/02; H04L 67/12

(52) U.S. Cl. CPC G01V 8/20; G05B 15/02; H04L 67/12

(57) ABSTRACT

The smart occupancy sensor system of the current disclosure is an instrument that is configured for use with an occupying space. The smart occupancy sensor system is a sensor that monitors the entry and exit of the occupying space. Specifically, the smart occupancy sensor system comprises a control system and at least one sensor. The sensor detects when a person enters or exits the occupying space. The sensor is attached to the control system such that the control system receives a signal from the sensor that indicates that a person has entered or exited the occupying space. The control system responds to the receipt of the signal by communicating with any of a plurality of smart devices positioned the occupying space.

### CROSS REFERENCE TO RELATED APPLICATIONS

This application claims the benefit of U.S. Provisional Patent Application Ser. No. 63/168,486 having a filing date of 31 Mar. 2021. The disclosure of the application above is incorporated herein by reference in its entirety.

### BACKGROUND OF THE DISCLOSURE

### Field of the Disclosure

Embodiments of the disclosure generally relates to the field of instrumentation and communications, more specifically, a smart occupancy sensor to detect and inventory occupying space conditions that are not elsewhere provided for.

### Description of the Related Art

In the prior art there is exists many attempts to provide a system and method to sense occupancy and manage the occupying space of a residence or building.

Some references, which may include patents, patent applications and various publications, are cited and discussed in the description of this disclosure. The citation and/or discussion of such references is provided merely to clarify the description of the present disclosure and is not an admission that any such reference is “prior art” to the disclosure described herein. The references cited and discussed in this specification are incorporated herein by reference in their entireties and to the same extent as if each reference was individually incorporated by reference.

### SUMMARY OF THE INVENTION

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a system including at least one sensor configured to detect motion of a plurality of humans positioned proximate an occupying space, the at least one sensor may include a housing having a front side, a first side and a second side, a first photoelectric sensor positioned on the first side of the housing and configured to emit a first electromagnetic beam from the front side, a second photoelectric sensor positioned on the second side of the housing and configured to emit a second electromagnetic beam from the front side, a microcontroller positioned within the housing and electrically coupled to the first photoelectric sensor and the second photoelectric sensor, and a communications module coupled to the microcontroller, a control system coupled to the at least one sensor may include a computer processor, a non-volatile memory storage device, and a sensor listening module configured to communicate with communications module. Other embodiments of this aspect include corresponding computer systems, apparatus, and computer programs recorded on one or more computer storage devices.

Implementations may include one or more of the following features. The system where the microcontroller is configured to determine a direction of motion of the plurality of humans relative to the occupying space in dependance of an interruption of the first electromagnetic beam and an interruption the second electromagnetic beam and is further configured to determine an occupancy of the occupying space based on the direction. The computer processor is configured to determine an entry event when the direction is into the occupying space and the computer processor is configured to determine an exit event is when the direction is out of the occupying space. The entry event is determined when the first electromagnetic beam is interrupted followed by both electromagnetic beams being interrupted, followed by only the second electromagnetic beam being interrupted and where the exit event is determined when the second electromagnetic beam is interrupted followed by both electromagnetic beams being interrupted, followed by only the first electromagnetic beam being interrupted. The communications module is configured to transmit a plurality of sensor signals to the sensor listening module based on the interruption of the of the first electromagnetic beam and the interruption of the second electromagnetic beam. The communications module may include a radio module, a plurality of ports and a power source and where the radio module is configured to transmit the sensor signals to the sensor listening module. The control system further may include an interaction module such as Home Assistant configured to communicate between the computer processor and a plurality of compatible smart devices. The plurality of compatible smart devices may include any of a smart phone, a tablet, a thermostat, a security system, a smart lock, an internet of things connected device, a computer, a network node, a wearable device, a game console, a storage device, a surveillance device, a printer, a scanner, a home voice assistant, a vehicle, a television and a home appliance. The control system is configured to control the plurality of compatible smart devices based at least in part on the occupancy of the occupying space. The at least one sensor may include a plurality of sensors where each of the plurality of sensors is positioned proximate each of the plurality of occupying spaces. The control system is configured to determine a building occupancy based on the occupancy of each of the plurality of occupying spaces. The control system is further configured to communicate with the plurality of compatible smart devices based on the building occupancy and the occupancy of each of the plurality of occupying spaces. The system may include a camera in electronic communication with the control system, and a facial recognition system in electronic communication with the control system configured, or other sensors used to determine an identity of at least one individual based on additional data points. The sensor may also contain additional sensors to detect other conditions of the occupying space including, but not limited to temperature, lighting conditions, and noise level and feed that data to the control system to create events based on specific conditions. The control system is further configured to communicate with the plurality of compatible smart devices based on the identity. The first photoelectric sensor may include a first infrared light source and a first light receiver and the second photoelectric sensor may include a second infrared light source and a second light receiver. The first infrared light source is configured to produce the first electromagnetic beam having a first beam angle and the second infrared light source is configured to produce the second electromagnetic beam having a second beam angle and where the first beam angle and the second beam angle are selected to cover a predetermined area of the occupying space. Implementations of the described techniques may include hardware, a method or process, or computer software on a computer-accessible medium.

One general aspect includes a sensor for detecting motion of a human. The sensor also includes a housing having a front side, a first side and a second side, a first photoelectric sensor positioned on the first side of the housing and configured to emit a first electromagnetic beam from the front side, a second photoelectric sensor positioned on the second side of the housing and configured to emit a second electromagnetic beam from the front side, and a microcontroller positioned within the housing and electrically coupled to the first photoelectric sensor and the second photoelectric sensor. Other embodiments of this aspect include corresponding computer systems, apparatus, and computer programs recorded on one or more computer storage devices, each configured to perform the actions of the methods.

Implementations may include one or more of the following features. The sensor where the microcontroller is configured to determine a direction of motion of the human in dependance of an interruption of the first electromagnetic beam and an interruption the second electromagnetic beam. A first direction is determined when the first electromagnetic beam is interrupted followed by both electromagnetic beams being interrupted, then finally only the second electromagnetic beam being interrupted and where a second direction is determined when the second electromagnetic beam is interrupted followed by both electromagnetic beams being interrupted, then finally only the first electromagnetic beam being interrupted. The sensor may include a communications module configured to transmit a plurality of sensor signals based on the interruption of the of the first electromagnetic beam and the interruption of the second electromagnetic beam. The communications module may include a radio module, a plurality of ports and a power source and where the radio module is configured to transmit the sensor signals to a computer processor. The first photoelectric sensor may include a first infrared light source and a first light receiver and the second photoelectric sensor may include a second infrared light source and a second light receiver. The first infrared light source is configured to produce the first electromagnetic beam having a first beam angle and the second infrared light source is configured to produce the second electromagnetic beam having a second beam angle and where the first beam angle and the second beam angle are selected to cover a predetermined area. Implementations of the described techniques may include hardware, a method or process, or computer software on a computer-accessible medium.

### BRIEF DESCRIPTION OF THE DRAWINGS

So that the manner in which the above-recited features of the present disclosure can be understood in detail, a more particular description of the disclosure, briefly summarized above, can be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 is a schematic diagram of a smart occupancy sensor system in accordance with the current disclosure;

FIG. 2 is a schematic diagram of a hub of a smart occupancy sensor system in accordance with the current disclosure;

FIG. 3 is a perspective view of a smart occupancy sensor in accordance with the current disclosure;

FIG. 4 is a schematic diagram of a smart occupancy sensor in accordance with the current disclosure;

FIG. 5 is a schematic diagram of a smart occupancy sensor in accordance with the current disclosure;

FIG. 6 is a graphical representation of an entry pattern of a smart occupancy sensor system in accordance with the current disclosure;

FIG. 7 is a graphical representation of an exit pattern of a smart occupancy sensor system in accordance with the current disclosure;

FIG. 8 is a schematic diagram of a smart occupancy sensor system in accordance with the current disclosure; and

FIG. 9 is a perspective view of a smart occupancy sensor in accordance with the current disclosure.

### DETAILED DESCRIPTION

In the following detailed description of the embodiments, reference is made to the accompanying drawings, which form a part hereof, and within which are shown by way of illustration specific embodiments by which the examples described herein can be practiced. It is to be understood that other embodiments can be utilized, and structural changes can be made without departing from the scope of the disclosure. For instance, as part of the present disclosure, examples will be given in terms of

All of the methods disclosed and claimed herein can be made and executed without undue experimentation in light of the present disclosure. While the apparatus and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. In addition, modifications may be made to the disclosed apparatus and components may be eliminated or substituted for the components described herein where the same or similar results would be achieved. All such similar substitutes and modifications apparent to those skilled in the art are deemed to be within the spirit, scope, and concept of the disclosure.

Although the invention(s) is/are described herein with reference to specific embodiments, various modifications and changes can be made without departing from the scope of the present invention(s), as presently set forth in the claims below. Accordingly, the specification and figures are to be regarded in an illustrative rather than a restrictive sense, and all such modifications are intended to be included within the scope of the present invention(s). Any benefits, advantages, or solutions to problems that are described herein with regard to specific embodiments are not intended to be construed as a critical, required, or essential feature or element of any or all the claims.

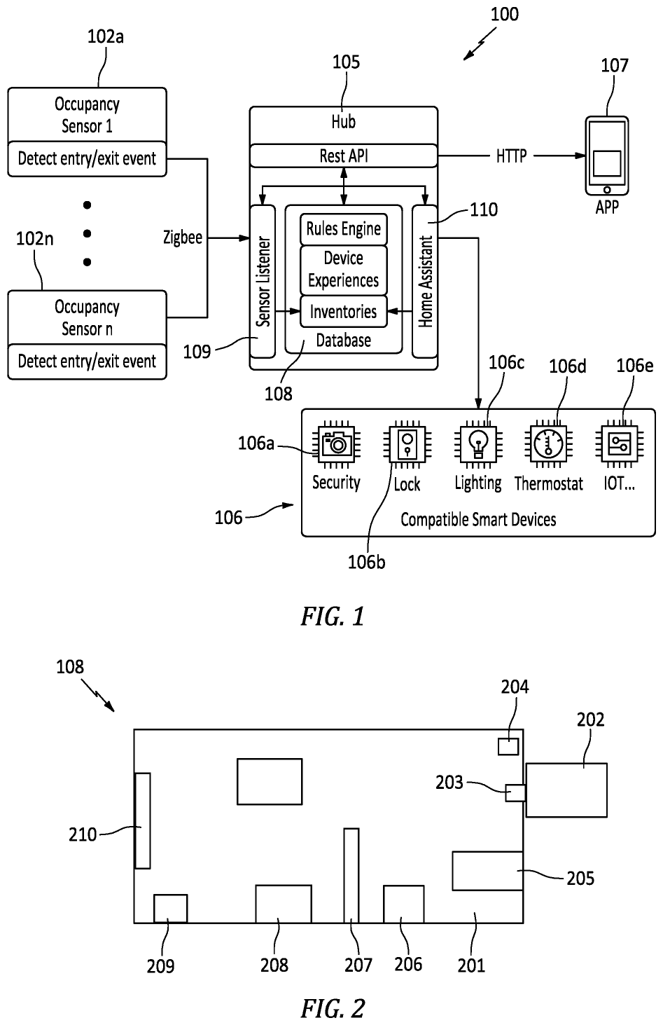

Referring to FIG. 1, smart occupancy sensor system 100 is an integrated system that is configured for use in monitoring a plurality of occupying spaces that can be occupied by humans. The occupying spaces can be rooms, offices, elevators, garages and the like. The smart occupancy sensor system 100 includes a plurality of sensors 102a-102n wherein the sensors monitor the entry and exit of persons with respect to specific occupying spaces. As will be explained in more detail herein below, sensor 102a can be mounted adjacent a doorway for a first occupying space to monitor the exit and entry of persons into and out of the first occupying space. Similarly, a plurality of sensors can be positioned to monitor the entry and exit of persons into and out of a respective occupying space and, for example, sensor 102n can be mounted adjacent a doorway for an n^{th} occupying space to monitor the exit and entry of persons into and out of the n^{th} occupying space. Sensor 102a can be mounted to a wall adjacent an opening to the first occupying space but it can be placed elsewhere proximate first occupying space as will be disclosed in more detail herein after. In addition to a plurality of sensors 102a-102n, smart occupancy sensor system 100 comprises a hub 105, a plurality of compatible smart devices 106 and a smart phone 107. Sensors 102a-102n can communicate with hub 105 via wireless communications protocols including a Zigbee radio signal.

Compatible smart devices 106 can include a security system 106a, door locking systems 106b, lighting systems 106c, HVAC controllers (thermostats) 106d and other internet of things (IOT) connected devices 106e. Compatible smart devices 106 can communicate with hub 105 via internet protocols such as Wi-Fi. Smart phone 107 can communicate with hub 105 via a number of wireless methods including Bluetooth®, radio and internet protocols such as Hypertext Transfer Protocol (HTTP). The plurality of smart devices 106 can further include a computer, a network node (e.g., home router), a wearable device (e.g., Fitbit, Apple Watch), a game console (e.g., XBox), storage devices (e.g., home NAS), surveillance devices (e.g., digital camera, IP camera), work related appliances (e.g., printer or scanner), home voice assistant, vehicle, media, television, home appliances (e.g., smart fridge, coffee maker), generic IoT devices (e.g., toothbrush) and the like.

Hub 105 includes a computer processor 108, such as a Raspberry Pi, capable of executing computer code and configured to control smart occupancy sensor system 100 and to communicate with, and appropriately control, a plurality of peripheral device including the plurality of sensors 102a-102n, the plurality of compatible smart devices 106 and smart phone 107. Hub 105 can include a radio communications device such as a Xbee radio module running a Zigbee wireless communications protocol configured to communicate with sensors 102a-102n. Hub 105 further includes an external power source and at least one application programming interface (API) as a connection between software running on computer processor 108 and software running on the other devices disclosed herein and part of smart occupancy sensor system 100. Hub 105 also includes a non-volatile memory storage device configured to host software such as a rules engine, databases that include device experiences and inventories of events. A device experience is a pre-programed set of rules to allow an Internet of Things (IoT) device to interact in a preconfigured manner to cater to human preferences. An example is a preset series of parameters to turn on smart lights to be dimmed if the automation is occurring in the middle of the night. Hub 105 includes sensor listening module 109 configured to receive signals from sensors 102a-102n and to communicate such signals to computer processor 108. Hub 105 further includes home assistant module 110 configured to communicate between computer processor 108 and compatible smart devices 106.

In its simplest form, hub 105 is an electronic device that: 1) receives communication from the plurality of sensors 102a-102n; 2) generates and sends commands to any of the plurality of compatible smart devices 106 based on the occupancy tracked by a plurality of sensors 102a-102n when predefined conditions are met, and 3) allows authorized users to administer and configure the system smart occupancy system 100 and plurality of sensors 102a-102n, and the plurality of compatible smart devices 106 using a smart phone 107. With reference to FIG. 2, there is shown some of the electrical componentry comprising hub 105 including computer processor 108 in the form of a Raspberry Pi. Connected to Raspberry Pi 108, a communications module 202 which can comprise a Xbee radio module connected via USB port 203, secondary USB port 204, ethernet port 205, audio port 206, camera serial interface 207, HDMI plug 208, power plug 209 and display serial interface 210.

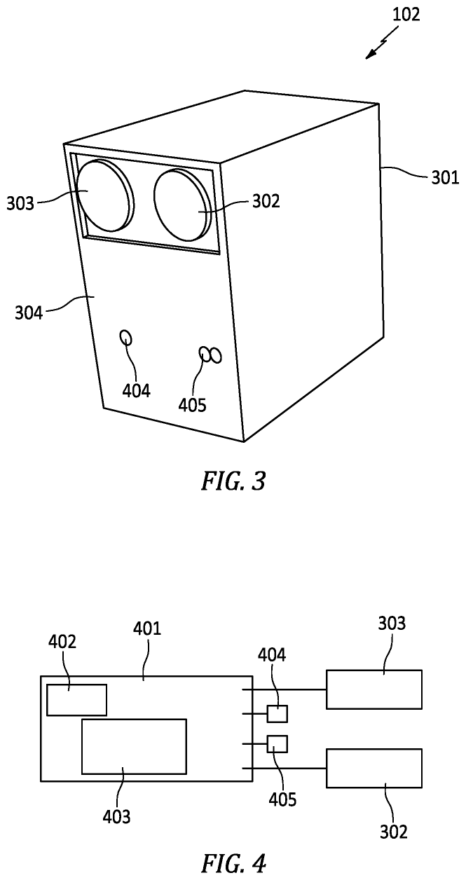

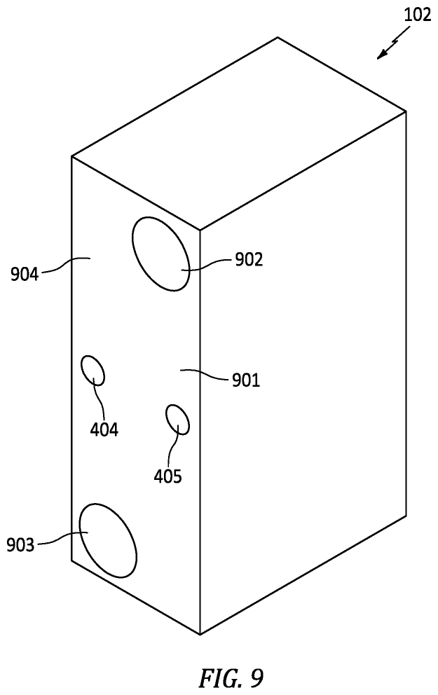

Referring now to FIG. 3, there is shown sensor 102 which comprises a housing 301, a first photoelectric sensor 302 and a second photoelectric sensor 303 positioned within the housing on a first side and a second side respectively and proximate a front side of housing 301. First photoelectric sensor 302 and the second photoelectric sensor 303 can include an infrared light source and a light receiver and in some embodiments of the present disclosure can comprise a diffuse photoelectric sensor model number E18-D80NK. Each of the first photoelectric sensor 302 and the second photoelectric sensor 303 are configured to collect emission and reception of the infrared light. Housing 301 is configured to mounted to a plurality of surfaces using any suitable technique such as adhesives, tapes, hook and loop fasteners and other fasteners. Housing 301 is also able to be embedded into a door frame as will be disclosed in more detail herein after with reference to FIG. 9. Referring now to FIG. 4, mounted within housing 301 are various components that comprise sensor 102. In some embodiments, such components include sensor circuit board 401, radio module 402, first photoelectric sensor 302 and second photoelectric sensor 303, micro-controller board 403, a first LED 404 and a second LED 405. First photoelectric sensor 302 and second photoelectric sensor 303 are as disclosed herein above and are electrically coupled to sensor circuit board 401. Radio module 402 can comprise a Xbee radio frequency module electrically connected to sensor circuit board 401 and configured to send sensor signals to the sensor listening module 109 (FIG. 1). Micro-controller 403 can comprise an Arduino Nano which is a microcontroller board. Micro-controller 403 comprises a plurality of digital input/output pins, analog inputs, a ceramic resonator, a USB connection, a power jack, and an in circuit serial programming header and a reset button. It can be powered using a USB cable, an AC-to-DC adapter or a battery. As will be appreciated by those skilled in the art, various resistors are also included in the components mounted to sensor circuit board 401. It should be appreciated by those skilled in the art that additional sensors can be added to detect specific conditions of the area the sensor is placed to be used in automation rules.

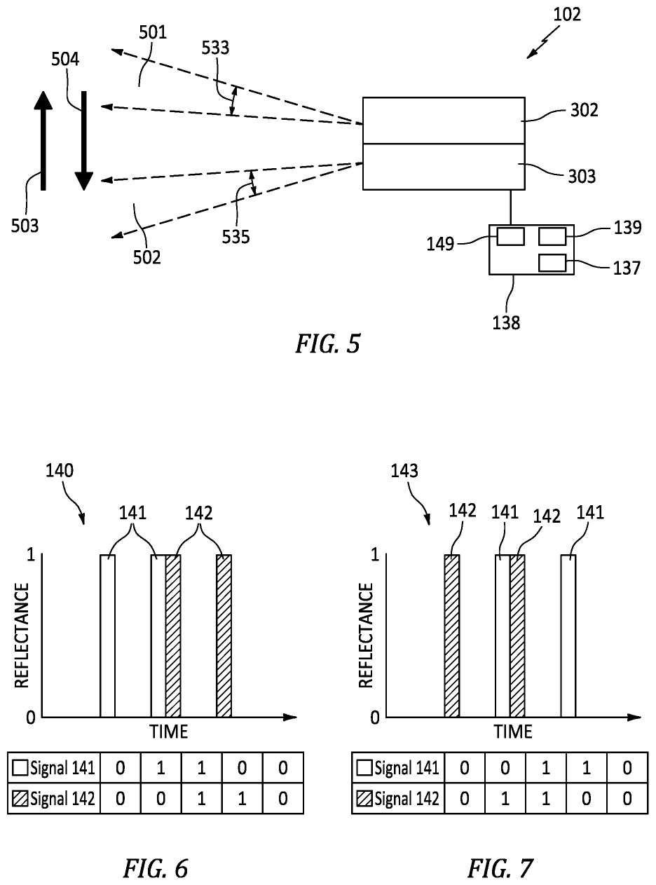

The operation of the control system 105, with reference to FIGS. 1 and 5, will now be disclosed with sensor 102 mounted in such a way to detect the entry and exit of a person from an occupancy space. With first photoelectric sensor 302 and second photoelectric sensor 303 positioned adjacent to one another as disclosed herein above, sensor 102 monitors the state of beam 501 from first photodetector 302 and the state of beam 502 from second photodetector 303 in order to detect whether a beam has been broken and restored by an individual passing through the pair of beams. As disclosed herein above, the photo electric sensors 302, 303 can comprise proximity-sensing type sensor arrangement wherein they transmit infrared radiation into an occupying space which are configured to reflect off of a person entering or exiting the occupying space and detectors in photo electric sensors 302, 303 detect this reflection. In this manner, the person entering or exiting the occupying space is detected when the receiver of first photoelectric sensor 302 and second photoelectric sensor 303 see a reflection of the transmitted sources rather than when they fail to see it. The person acts as the reflector so that detection of light is reflected off the person. The source of first photoelectric sensor 302 sends out a beam of light 501 (which can be infrared, visible red, or laser) that diffuses into a cone in all directions at beam angle 533, filling a predetermined area of the occupying space. The source of second photoelectric sensor 303 sends out a beam of light 502 (again, which can be infrared, visible red, or laser) that diffuses into a cone in all directions at beam angle 535, filling a portion of occupying space. Sensor 102 can include a light baffle (not shown), which comprises a non-light transmittable material positioned in a vertical direction relative to occupying space. First photoelectric sensor 302 and second photoelectric sensor 303 can be positioned at angle relative to each other such that the cone of light 501 and the cone of light 502 do not interfere with each other. In some embodiments, this angle is approximately 30 degrees. The aforementioned light baffle can be positioned between first photoelectric sensor 302 and second photoelectric sensor 303 such that the cone of light 501 and the cone of light 502 are truncated in the vertical plane. In such an arrangement, the light baffle prevents light 501 and light 502 from overlapping. The first photoelectric sensor 302 and second photoelectric sensor 303 use light 501 and light 502 as a sequential tripwire that detects a person that passes through the electromagnetic beams 501, 502.

Still referring to FIG. 5, and for purposes of an example, an entry into an occupying space is designated as entry direction 504 and an exit from the same occupying space is designated as exit direction 503. If there is an enter or exit event, sensor 102 will send a message appropriate to the type of event to hub 105 to raise an event. If the sensor 102 has not been tripped, the sensor 102 continues to monitor whether the beams 501, 502 have been broken. If the computer processor 108 receives notification that the sensor 102 has been tripped, the computer processor 108 receives identification of the room exited, and identification of the room entered as configured on the sensor 102. The computer processor 108 can then use a rules engine and determine actions to take, or monitor for and raise as time passes, on smart devices 106 based on user preferences stored in the memory of computer processor 108 for the occupying space. Computer processor 108 can further determine whether the occupying space(s) of interest is empty and tallies how many persons are within the occupying space(s). In addition, the computer processor 108 can track the number of individuals inside of a building based on adding up all occupying spaces. Smart occupancy sensor system 100 can also remember a person who is in a room even if the sensor 102 cannot detect their motion (ie not moving while sleeping, around a corner, in a walk in closet, etc).

As disclosed herein above, first photoelectric sensor 302 and second photoelectric sensor 303 are sequential electronic trip wires that are actuated by the reflection of light from electromagnetic beams 501 and 502. The sensor 102 sends an event to the hub 105. The smart occupancy sensor system 100 can comprise a modular design allowing more sensors to pass data about the occupancy detection, such as how much light is currently in the room, amount of noise, devices sensed as a user passed by for preference identification. Other embodiments include a power savings mode that can be enabled by detecting motion with a standard proximity monitor and enable additional sensors to power on only when an individual may be about to enter or exit a particular room.

In operation, and still referring to FIG. 5, sensor 102 determines an entry event or an exit event in dependance upon the interruption of electromagnetic beam 501 and 502. When a person travels in the entry 504 direction and enters an occupying space after being in a state where electromagnetic beam 501 and 502 are not reflecting to photoelectric sensor 302 or 303 respectively, electromagnetic beam 501 is reflected back to first photoelectric sensor 302 at a first instance of time without electromagnetic beam 502 reflecting to photoelectric sensor 303, followed by both electromagnetic beams 501 and 502 which are reflected back to 302 and 303 respectively, followed by electromagnetic beam 502 reflected back to second photoelectric sensor 303 without electromagnetic beam 501 reflecting back to photoelectric sensor 302 at a third instance of time, and finally no reflection from electromagnetic beam 501 or 502 to photoelectric sensors 302 and 303 respectively at the fourth instance of time. The entry pattern 140 of signal generation is best seen with reference to FIG. 6 wherein signal 141 is the reflectance at the first photoelectric sensor 302 and wherein signal 142 is the reflectance at the second photoelectric sensor 303. When a person travels in the exit 503 direction and exits the occupying space after being in a state where electromagnetic beam 501 and 502 are not reflecting back to photoelectric sensor 302 and 303 respectively, followed by electromagnetic beam 502 is reflected back to second photoelectric sensor 303 while electromagnetic beam 501 is not reflected back to photoelectric sensor 302, followed by a second instance of time, electromagnetic beam 502 and 501 are both reflected back to photoelectric sensor 303 and 302 respectively, followed by a third instance of time electromagnetic beam 502 is not reflected back to first photoelectric sensor 303 while electromagnetic beam 501 is reflected back to photoelectric sensor 302, and finally no reflections from electromagnetic beam 502 and 501 back to photoelectric sensors 303 and 302 respectively. The exit pattern 143 of signal generation is best seen with reference to FIG. 7 wherein signal 142 is the reflectance at the second photoelectric sensor 303 and wherein signal 141 is the reflectance at the first photoelectric sensor 302. Computer processor 108 receives confirmation that sensor 102 observed entry pattern 140 and exit pattern 143 and keeps a tally of the number of persons in the occupying space, including whether the occupying space is occupied or unoccupied and communicates with smart devices 106 according to instructions provided by a user as will be described in more detail herein after.

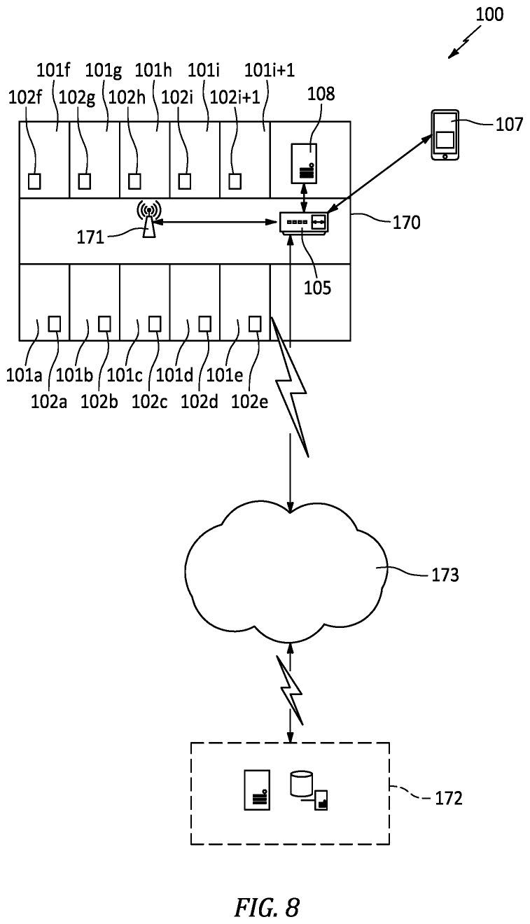

Referring now to FIG. 8, there is shown an embodiment of smart occupancy sensor system 100 configured for building 170 to determine a building occupancy and other parameters related to building occupancy. Building 170 includes a plurality of occupying spaces 101a-101i+1 with each occupying space including a corresponding sensor 102a-102i+1 positioned therein. Although not shown, each of the occupying spaces 101a-101i+1 includes any of the plurality of smart devices 106. In this particular embodiment, sensors 102a-102i+1 monitoring the entry and exit of persons into and out of the respective occupying spaces 101a-101i+1 as disclosed herein above and communicate those signals to control system 105 via Zigbee mesh network 171 established by the plurality of smart devices. Two way communication is configured between hub 105 and computer 108 as well as smart phone 107 and other smart hubs used for home automation. Some embodiments of smart occupancy sensor system 100 further include hub 105 connected to remote computing equipment 172 via the cloud 173. It should be appreciated by those skilled in the art that embodiments of the present disclosure include the ability to add multiple sensors to a room with multiple access points.

In some embodiments of the smart occupancy sensor system 100, a user can use an application on smart phone 107 or computer 108 to map out the plurality of occupying spaces 101a-101i+1 in the building 170 (or home) and identify where the sensors 102a-102i+1 or virtual trip wires demark one occupying space from another and communicate. The sensors 102a-102i+1 will trigger an event, a person exiting or entering, and communicate to the hub 105 when a person moves from one room to another. An additional sensor can be positioned to determine when people leave the home through an exit or enter the home through an entrance.

As disclosed herein above, hub 105 is configured to track the count of people as they enter and exit an occupying space and trigger events to follow user defined logic and interact with the plurality of smart devices 106. In the event a user is not properly counted moving from one room to another, the hub 105 can be overridden by a user through the app, or a smart assistant. Preprogramed events can be made available to the users, or users can be able to create custom events based on logic they enter into the smart occupancy sensor system 100. For instance, smart occupancy sensor system 100 can be configured such that the first person entering an unoccupied occupying space triggers different events than the second entering the same occupying space with somebody already occupying the room. For instance, the communication to a smart lighting device can be a command to not turn on the lights to the brightest setting if somebody is already in the room and turned them off to go to sleep if the occupying space is a bedroom. Smart occupancy sensor system 100 can be configured to cooperate with other sensors and smart devices such as light sensors to determine the brightness within a room and then to adjust lighting 106c in accordance with predetermined rules. In another example, Smart occupancy sensor system 100 can be configured to enable security system 106a to command a smart lock to lock preselected doors after all people have exited a house and can further include a delay before the doors are locked. Smart occupancy sensor system 100 can be further configured to communicate with thermostat 106d based on which rooms of a building are occupied and to send an alert to smart phone 107 if an IOT enabled appliance, such as a stove, is left on after the building is no longer occupied. In a commercial setting, smart occupancy sensor system 100 can be configured to optimize elevators to only stop on a floor when there is room for additional passengers; free up a conference room that was reserved but remains empty or alert when a conference room is over max capacity; and route customers to other occupying spaces based on specific areas being crowed.

Embodiments of the present disclosure enable novel capabilities for home automation technologies by tracking occupancy of an occupying space, such as alerts and notifications when room occupancies go over limits, and events when all the people leave a particular occupying space. A user can further control how room to room interactions will be handled for example, don't immediately turn a smart lighting system on and/or off when a sensor 102 detects an event. Instead, smart occupancy sensor system 100 can be configured to allow a smart lighting system 106c to fade, or to have a delayed reaction time. In such embodiments, smart occupancy sensor system 100 is configured to resemble how a human would want the environment within occupying spaces to flow naturally. Smart occupancy sensor system 100 can also be configured to keep track of the occupancy of an entire building and enable additional events (such as when a building is empty) to enable a security system 106a.

Referring now to FIG. 9, there is shown an alternative embodiment of sensor 102 which comprises a housing 901, a first photoelectric sensor 302 and a second photoelectric sensor 903 positioned within the housing on a first side and a second side respectively and proximate a front side 304 of housing 901. Similar to that sensor disclosed with reference to FIG. 3, first photoelectric sensor 902 and the second photoelectric sensor 903 can include an infrared light source and a light receiver and in some embodiments of the present disclosure can comprise a diffuse photoelectric sensor model number E18-D80NK. Each of the first photoelectric sensor 902 and the second photoelectric sensor 903 are configured to collect emission and reception of the infrared light. Sensor 102 further includes LEDs 405, 406. It should be appreciated by those skilled in the art that housing 901 is configured to mounted within a space, such as a door frame at an entry/exit location of an occupying space. Housing 301 is also able to be embedded into a door frame as will be disclosed in more detail herein after with reference to FIG. 9.

In some embodiments, smart occupancy sensor system 100 can be configured to use other devices to obtain the identity of a specific person and use that information to track that person throughout a building. Such other devices include smart assistants, facial recognition systems and the like. The facial recognition systems can interface with a digital camera to capture an image of an individual. Embodiments of current disclosure further include devices that can track and identify specific individuals through their electronic “footprint” including, but not limited to, nearfield communications, a wireless technology system such as Bluetooth®, Wi-Fi and the like. In such embodiments smart occupancy sensor system 100 can be configured to customize the compatible smart devices 106 to the identified individual. For example, when a specific individual is identified, hub 105 can play preselected music and “follow” the identified person around the house by activating speakers in rooms upon entry and deactivating music in rooms upon exit.

Unless stated otherwise, terms such as “first” and “second” are used to arbitrarily distinguish between the elements such terms describe. Thus, these terms are not necessarily intended to indicate temporal or other prioritization of such elements. The terms “coupled” or “operably coupled” are defined as connected, although not necessarily directly, and not necessarily mechanically. The terms “a” and “an” are defined as one or more unless stated otherwise. The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system, device, or apparatus that “comprises,” “has,” “includes” or “contains” one or more elements possesses those one or more elements but is not limited to possessing only those one or more elements.

While the foregoing is directed to embodiments of the present disclosure, other and further embodiments of the disclosure may be devised without departing from the basic scope thereof, and the scope thereof is determined by the claims that follow.

What is claimed is:

1. A system, comprising:
at least one sensor configured to detect motion of a plurality of humans positioned proximate an occupying space, the at least one sensor comprising:
a housing having a front side, a first side and a second side;
a first photoelectric sensor positioned on the first side of the housing and configured to emit a first electromagnetic beam from the front side;
a second photoelectric sensor positioned on the second side of the housing and configured to emit a second electromagnetic beam from the front side;
a microcontroller positioned within the housing and electrically coupled to the first photoelectric sensor and the second photoelectric sensor; and
a communications module coupled to the microcontroller;
a control system coupled to the at least one sensor comprising:
a computer processor;
a non-volatile memory storage device; and
a sensor listening module configured to communicate with communications module.

2. The system of claim 1 wherein the microcontroller is configured to determine a direction of motion of the plurality of humans relative to the occupying space in dependance of an interruption of the first electromagnetic beam and an interruption the second electromagnetic beam and is further configured to determine an occupancy of the occupying space based on the direction.

3. The system of claim 2 wherein the computer processor is configured to determine an entry event when the direction is into the occupying space and the computer processor is configured to determine an exit event is when the direction is out of the occupying space.

4. The system of claim 3 wherein the entry event is determined when the first electromagnetic beam is interrupted at first period of time, followed by the first electromagnetic beam and the second electromagnetic beam being interrupted at a second period of time, followed by the second electromagnetic beam being interrupted at a third period of time, followed by the first electromagnetic beam and second electromagnetic beam being uninterrupted at a third period of time and wherein the exit event is determined when the second electromagnetic beam is interrupted at a fourth period of time, followed by the first electromagnetic beam and the second electromagnetic beam being interrupted at a fifth period of time, followed by the first electromagnetic beam being interrupted at a sixth period of time, followed by the first electromagnetic beam and second electromagnetic beam being uninterrupted at a seventh period of time.

5. The system of claim 3 wherein the communications module is configured to transmit a plurality of sensor signals to the sensor listening module based on the entry or exit event.

6. The system of claim 5 wherein the communications module comprises a radio module, a plurality of ports and a power source and wherein the radio module is configured to transmit the sensor signals to the sensor listening module.

7. The system of claim 6 wherein the control system further comprises an interaction module configured to communicate between the computer processor and a plurality of compatible smart devices.

8. The system of claim 7 wherein the plurality of compatible smart devices comprises any of a smart phone, a tablet, a thermostat, a security system, a smart lock, an internet of things connected device, a computer, a network node, a wearable device, a game console, a storage device, a surveillance device, a printer, a scanner, a home voice assistant, a vehicle, a television and a home appliance.

9. The system of claim 8 wherein the control system is configured to control the plurality of compatible smart devices based at least in part on the occupancy of the occupying space.

10. The system of claim 7 further comprising a building including a plurality of occupying spaces and wherein the at least one sensor comprises a plurality of sensors wherein each of the plurality of sensors is positioned proximate each of the plurality of occupying spaces.

11. The system of claim 10 wherein the control system is configured to determine a building occupancy based on the occupancy of each of the plurality of occupying spaces.

12. The system of claim 11 wherein the control system is further configured to communicate with the plurality of compatible smart devices based on the building occupancy and the occupancy of each of the plurality of occupying spaces.

13. The system of claim 1 wherein the first photoelectric sensor comprises a first infrared light source and a first light receiver and the second photoelectric sensor comprises a second infrared light source and a second light receiver.

14. The system of claim 13 wherein the first infrared light source is configured to produce the first electromagnetic beam having a first beam angle and the second infrared light source is configured to produce the second electromagnetic beam having a second beam angle and wherein the first beam angle and the second beam angle are selected to cover a predetermined area of the occupying space.

15. The system of claim 12 further comprising a device configured to determine an identity of at least one individual.

16. The system of claim 15 wherein the control system is further configured to communicate with the plurality of compatible smart devices based on the identity.

17. A sensor for detecting motion of a human, the sensor comprising:
a housing having a front side, a first side and a second side;
a first photoelectric sensor positioned on the first side of the housing and configured to emit a first electromagnetic beam from the front side;
a second photoelectric sensor positioned on the second side of the housing and configured to emit a second electromagnetic beam from the front side; and
a microcontroller positioned within the housing and electrically coupled to the first photoelectric sensor and the second photoelectric sensor.

18. The sensor of claim 17 wherein the microcontroller is configured to determine a direction of motion of the human in dependance of an interruption of the first electromagnetic beam and an interruption the second electromagnetic beam.

19. The sensor of claim 18 wherein a first direction is determined when the first electromagnetic beam is interrupted at first period of time, followed by the first electromagnetic beam and the second electromagnetic beam being interrupted at a second period of time, followed by the second electromagnetic beam being interrupted at a third period of time, followed by the first electromagnetic beam and second electromagnetic beam being uninterrupted at a third period of time and wherein a second direction is determined when the second electromagnetic beam is interrupted at a fourth period of time, followed by the first electromagnetic beam and the second electromagnetic beam being interrupted at a fifth period of time, followed by the first electromagnetic beam being interrupted at a sixth period of time, followed by the first electromagnetic beam and second electromagnetic beam being uninterrupted at a seventh period of time.

20. The sensor of claim 19 further comprising a communications module configured to transmit a plurality of sensor signals based on the interruption of the of the first electromagnetic beam and the interruption of the second electromagnetic beam.

21. The sensor of claim 20 wherein the communications module comprises a radio module, a plurality of ports and a power source and wherein the radio module is configured to transmit the sensor signals to a computer processor.

22. The sensor of claim 17 wherein the first photoelectric sensor comprises a first infrared light source and a first light receiver and the second photoelectric sensor comprises a second infrared light source and a second light receiver.

23. The sensor of claim 22 wherein the first infrared light source is configured to produce the first electromagnetic beam having a first beam angle and the second infrared light source is configured to produce the second electromagnetic beam having a second beam angle and wherein the first beam angle and the second beam angle are selected to cover a predetermined area.

24. The system of claim 16 wherein the device configured to determine an identity of at least one individual is configured to track an electronic footprint of the at least one individual and is comprised any of a facial recognition system, a near field communications system, a wireless technology system and a Wi-Fi system. configured to determine an identity of at least one individual based on an image from the camera.
